(19) United States
(12) Patent Application Publication
(10) Pub. No.: US 2006/0078597 A1
(43) Pub. Date: Apr. 13, 2006

(54) Ascorbic acid salt suspensions and use thereof as antioxidants

(75) Inventors: Axel Jentzsch, Mannheim (DE); Andreas Habich, Speyer (DE); Christian Kopsel, Weinheim (DE); Andreas Ernst, Worms (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/520,533

(22) PCT Filed: Jul. 16, 2003
(86) PCT No.: PCT/EP03/07705

§ 371 Date: Jan. 6, 2005

(30) Foreign Application Priority Data
Jul. 24, 2002 (DE) 102 33 738.1
Jan. 29, 2003 (DE) 103 03 527.3

(51) Int. Cl. A61K 47/00; A23K 1/165; A61K 31/355; A61K 31/375; A61K 31/015

(52) U.S. Cl. 424/442; 514/474; 514/763; 514/458; 426/635

(57) ABSTRACT

Suspensions comprising A) at least one oxidation-sensitive substance selected from the group consisting of carotenoids, retinoids and unsaturated fatty acids and B) solid particles of one or more salts of ascorbic acid in a dispersant in which the salts of ascorbic acid are insoluble.

The present invention relates to suspensions of salts of ascorbic acid, to the preparation and use thereof as antioxidants in cosmetics, pharmacy and in human and animal nutrition.

The oxidation of fats and oils and of lipophilic active substances such as, for example, carotenoids and retinoids is often a serious problem in the production and storage of cosmetic and pharmaceutical preparations and of human foods and animal feeds. Oxidation of fats and oils results inter alia in rancidity of human and animal foods and of cosmetic and pharmaceutical preparations, which ordinarily leads to these products being unusable.

Oxidation of active substances such as carotenoids which are widely used as coloring pigments in the food and feed sectors may lead to unwanted degradation reactions and to a reduction or loss of the intensity of the carotenoid color.

The class of retinoids comprises some of the most active substances employed in cosmetics and dermatology. They act inter alia to regulate normal cell growth and influence the differentiation of epithelial cells. Thus, retinoic acids are employed for the treatment of acne and retinol is employed for example in antiwrinkle creams.

However, there are likewise great restrictions on the use of retinoids, attributable inter alia to the great instability of the compounds. This is why strict precautionary measures must be observed during the production of retinoid-containing preparations. For example, the production must take place entirely under protective gas, and the packaging for the finished product must be impermeable to oxygen.

An antioxidant effect against this is provided by exclusion of light and oxygen, addition of heavy metal-complexing substances such as, for example, citrates, gallates or tartrates, and addition of natural (vitamin E, vitamin C, carnosine, gallates) or synthetic (butylated hydroxyanisole, butylated hydroxytoluene) antioxidants.

The use of the abovementioned synthetic antioxidants is not always unobjectionable from the viewpoints of toxicology and—because of their lack of biodegradability—ecology.

A number of processes for stabilizing retinoids have been described. Thus, EP-A-1 055 720 discloses the stabilization of oxygen-sensitive compounds by using thio compounds or glycoproteins with exclusion of oxygen.

WO 93/00085 and EP-A-0 440 398 disclose the use both of water-soluble and of fat-soluble antioxidants together with chelating agents for stabilizing retinoids.

EP-A-0 590 029 describes suspensions of ascorbic acid particles in the micrometer range and their use as antioxidants inter alia for fats, oils, carotenoids and human foods.

WO 01/67896 describes a process for preparing oily suspensions of solid particles of water-soluble vitamins by grinding these particles until the average particle size is from 0.1 to 100 μm.

However, the described processes do not always lead to adequate stabilization within the meaning of the invention. On the contrary, both water-soluble antioxidants, especially ascorbic acid, and fat-soluble antioxidants, especially tocopherol, show a destabilizing effect in connection with retinoids in certain concentration ranges.

In addition, on use of certain antioxidant combinations together with retinoids there may in some circumstances be unwanted side effects, e.g. yellowish discoloration of the preparations which makes these systems unusable in cosmetics or in the food sector.

It is an object of the present invention to provide an antioxidant composition which does not have the abovementioned disadvantages of the prior art.

We have found that this object is achieved by suspensions comprising
A) at least one oxidation-sensitive substance selected from the group consisting of carotenoids, retinoids and unsaturated fatty acids and
B) solid particles of one or more salts of ascorbic acid in a dispersant in which the salts of ascorbic acid are insoluble.

Carotenoids mean according to the invention the following compounds: β-carotene, lycopene, lutein, astaxanthin, zeaxanthin, cryptoxanthin, citranaxanthin, canthaxanthin, bixin, β-apo-4-carotenal, β-apo-8-carotenal, β-apo-8-carotenoic esters, singly or as mixture. Carotenoids which are preferably used are β-carotene, lycopene, lutein, astaxanthin, zeaxanthin, citranaxanthin and canthaxanthin.

The carotenoids can moreover be employed in crystalline form or as formulation—for example as dry powder as disclosed in EP-A-0 065 193.

The preferred use in the case of lycopene, astaxanthin and canthaxanthin is of lycopene-, astaxanthin- and canthaxanthin-containing dry powders, for example LycoVit®, Lucantin® Pink and Lucantin® Red (10% dry powders respectively of lycopene, astaxanthin and canthaxanthin, from BASF AG, Ludwigshafen, Germany) together with the salts of ascorbic acid.

Retinoids mean for the purposes of the present invention vitamin A alcohol (retinol) and its derivatives such as vitamin A aldehyde (retinal), vitamin A acid (retinoic acid) and vitamin A esters (for example retinyl acetate, retinyl propionate and retinyl palmitate). The term retinoic acid covers in this connection both all-trans-retinoic acid and 13-cis-retinoic acid. The terms retinol and retinal preferably comprise the all-trans compounds. The retinoid preferably used for the suspensions of the invention is all-trans-retinol, referred to as retinol hereinafter.

Examples of unsaturated fatty acids are undecylenic acid, palmitoleic acid, oleic acid, linoleic acid, linolenic acid, arachidonic acid, eicosapentaenoic acid, docosahexaenoic acid.

The term ascorbic acid encompasses both L-ascorbic acid and its diastereomeric form, D-ascorbic acid (isoascorbic acid). The salts of L-ascorbic acid are used as preferred embodiment of the solid particles of the invention.

Examples of salts of L-ascorbic acid are alkali metal or alkaline earth metal salts of L-ascorbic acid such as sodium L-ascorbate, potassium L-ascorbate or calcium L-ascorbate, but also salts of L-ascorbic acid with organic amine compounds such as choline ascorbate or L-carnitine ascorbate. Alkali metal salts of L-ascorbic acid are preferably used, particularly preferably sodium L-ascorbate. The term ascorbic acid or ascorbate stands hereinafter for the L form.

The abovementioned salts of ascorbic acid can be employed both in crystalline form with a purity of more than 95%, preferably more than 98%, particularly preferably more than 99%, and in formulated form, for example as granules, beadlet or as spray-dried powder. The abovementioned salts in their crystalline form are preferred.

Suitable dispersants in which the salts of ascorbic acid are insoluble are generally those substances in which the solubility of the ascorbate salts is less than 0.01 g/g of dispersant. These include, for example, fats, oils, waxes and organic solvents such as ethers, petroleum ethers, aromatic hydrocarbons such as toluene or xylene, chlorinated hydrocarbons such as dichloromethane or chloroform.

The dispersants in which the salts of ascorbic acid are insoluble and which are preferably used for the purposes of the present invention mean oils suitable for use in cosmetics and in human or animal nutrition.

Examples of suitable cosmetic oily substances are guerbet alcohols based on fatty alcohols having 6 to 18, preferably 8 to 10, carbon atoms, esters of linear C₆-C₂₂ fatty acids with linear C₆-C₂₂ fatty alcohols, esters of branched C₆-C₁₃ carboxylic acids with linear C₆-C₂₂ fatty alcohols, esters of linear C₆-C₂₂ fatty acids with branched alcohols, especially 2-ethylhexanol, esters of hydroxycarboxylic acids with linear or branched C₆-C₂₂ fatty alcohols, in particular dioctyl malate, esters of linear and/or branched fatty acids with polyhydric alcohols (such as, for example, propylene glycol, dimerdiol or trimertriol) and/or guerbet alcohols, triglycerides based on C₆-C₁₀ fatty acids, liquid mono/di/triglyceride mixtures based on C₆-C₁₈ fatty acids, esters of C₆-C₂₂ fatty alcohols and/or guerbet alcohols with aromatic carboxylic acids, in particular benzoic acid, vegetable oils, branched primary alcohols, substituted cyclohexanes, linear and branched C₆-C₂₂ fatty alcohol carbonates, Guerbet carbonates, esters of benzoic acid with linear and/or branched C₆-C₂₂ alcohols (e.g. Finsolv® TN), linear or branched, symmetrical or asymmetrical dialkyl ethers having 6 to 22 carbon atoms per alkyl group, ring-opened products of epoxidized fatty acid esters with polyols, silicone oils and/or aliphatic or naphthenic hydrocarbons.

Examples of suitable silicone compounds are dimethylpolysiloxanes, methylphenylpolysiloxanes, cyclic silicones and amino-, fatty acid-, alcohol-, polyether-, epoxy-, fluorine-, glycoside- and/or alkyl-modified silicone compounds which may be both liquid and resinous at room temperature. Typical examples of fats are glycerides, and suitable waxes are, inter alia, beeswax, carnauba wax, candelilla wax, montan wax, paraffin wax or microwaxes, where appropriate in combination with hydrophilic waxes, e.g. cetylstearyl alcohol or partial glycerides. Stabilizers which can be employed are metal salts of fatty acids such as, for example, magnesium, aluminum and/or zinc stearate.

Suitable edible oils are ordinarily all physiologically acceptable oils—of both vegetable and animal origin—especially those oils which are liquid at 20° C. or which form, alone or together with other oils, the liquid phase in the suspension at 20° C. Those which should be preferably mentioned in this connection are sunflower oil, palm oil, sesame oil, corn oil, cottonseed oil, soybean oil or peanut oil, esters of medium chain-length triglycerides and, in addition, fish oils such as, for example, mackerel, sprat or salmon oil. Particularly preferred for animal nutrition are fish oils, corn oil, sunflower oil and peanut oil. Additionally advantageous for the foods/drugs/cosmetics sectors are the esters of medium chain-length triglycerides (e.g. Delios® SK from Grünau; n-octanoic acid/n-decanoic acid triglyceride).

The amount of the abovementioned oxidation-sensitive substances in the suspensions of the invention is in the range from 0.1 to 40% by weight, preferably from 0.5 to 20% by weight, particularly preferably from 1 to 15% by weight, very particularly preferably in the range from 3 to 12% by weight, where the data in % by weight are based on the total amount of the suspensions.

The content of one or more of the abovementioned salts of ascorbic acid is in the range from 1 to 50% by weight, preferably from 5 to 40% by weight, particularly preferably from 10 to 35% by weight, very particularly preferably in the range from 15 to 25% by weight, where the data in % by weight are based on the total amount of the suspensions.

Preferred suspensions for the purpose of the present invention comprise
A) at least one retinoid, particularly preferably retinol, and
B) solid particles of one or more alkali metal and/or alkaline earth metal salts of ascorbic acid, particularly preferably sodium ascorbate.

Likewise preferred are suspensions comprising as component B) solid particles of one or more salts of ascorbic acid whose average particle size D[4.3] is in the range from 0.01 to 1000 μm, preferably from 0.1 to 500 μm, particularly preferably from 0.2 to 200 μm, very particulary preferably in the range from 0.5 to 50 μm, especially 0.5 to 20 μm. The term D[4.3] refers to the volume-weighted average diameter (see handbook for Malvern Mastersizer S, Malvern Instruments Ltd., UK).

The suspensions of the invention may additionally comprise vitamin E, vitamin E derivatives or mixtures thereof. The designation vitamin E stands in this connection for natural or synthetic α-, β-, γ- or δ-tocopherol and for tocotrienol. Examples of vitamin E derivatives are tocopheryl C₁-C₂₀ alkanoic esters such as tocopheryl acetate or tocopheryl palmitate. Natural or synthetic α-tocopherol is preferably used. Vitamin E is employed in an amount of from 1 to 50% by weight, preferably from 1 to 40% by weight, particularly preferably from 5 to 30% by weight, very particularly preferably from 15 to 25% by weight (data in % by weight based on the total amount of the suspensions).

It is also possible to use vitamin E, vitamin E derivatives or mixtures thereof as dispersants for the suspensions of the invention.

In this case, the suspensions may comprise
A) 0.1 to 40% by weight of one or more oxidation-sensitive substances selected from the group consisting of carotenoids, retinoids and unsaturated fatty acids and
B) 1 to 50% by weight of one or more salts of ascorbic acid and
C) 10 to 98.9% by weight of vitamin E, vitamin E derivatives or mixtures thereof,
   where the % by weight data are based on the total amount of the suspension, and the total of the proportions of A to C by weight is 100%.

It may in some cases be advantageous for the suspensions of the invention additionally to comprise at least one desiccant, at least one thickener and/or at least one surface-active agent.

Examples of suitable thickeners are polysaccharides, in particular xanthan gum, guar-guar, agar-agar, alginates and Tyloses, carboxymethylcellulose and hydroxyethylcellulose, also high molecular weight polyethylene glycol monoesters and diesters of fatty acids, polyacrylates (e.g. Carbopols® from Goodrich or Synthalens® from Sigma), polyacrylamides, polyvinyl alcohol (PVA) and polyvinylpyrrolidone (PVP) and VA/VP copolymers (Luviskol®, from BASF), polyethylenes, surfactants such as, for example, ethoxylated fatty acid glycerides (inter alia Cremophor® CO from BASF), esters of fatty acids with polyols such as, for example, pentaerythritol or trimethylolpropane, fatty alcohol ethoxylates with restricted homolog distribution or alkyl oligoglucosides, electrolytes such as sodium chloride and ammonium chloride, and silicates such as magnesium aluminum silicates (Veegum® Ultra from R.T. Vanderbilt Comp.,Inc.), sheet silicates such as Bentone® EW (from Elementis Specialties) or Tixogel® VP (from Süd Chemie). Further thickeners are colloidal silica (Aerosil® from Degussa), polyethylene glycols (Lutrol® from BASF), stearic acid, trihydroxystearin, polyoxyethylene glycerol triricinoleate, triglycerides of C₁₂/C₁₄ fatty acids.

The amount of thickener in the suspensions of the invention is in the range from 0.1 to 10% by weight, preferably 0.5 to 8% by weight, particularly preferably from 0.7 to 3% by weight, very particularly preferably from 0.8 to 1.5% by weight based on the total amount of the suspensions.

Examples of suitable surface-active agents are the following substances:
1) adducts of from 2 to 30 mol of ethylene oxide and/or 0 to 5 mol of propylene oxide with linear fatty alcohols having 8 to 22 C atoms, with fatty acids having 12 to 22 C atoms and with alkylphenols having 8 to 15 C atoms in the alkyl group;
2) C12/18 fatty acid monoesters and diesters of adducts of from 1 to 30 mol of ethylene oxide with glycerol;
3) glycerol monoesters and diesters and sorbitan monoesters and diesters of saturated and unsaturated fatty acids having 6 to 22 carbon atoms and the ethylene oxide adducts thereof;
4) alkyl monoglycosides and oligoglycosides having 8 to 22 carbon atoms in the alkyl radical and the ethoxylated analogs thereof;
5) adducts of from 15 to 60 mol of ethylene oxide with castor oil and/or hardened castor oil;
6) polyol esters and, in particular, polyglycerol esters such as, for example, polyglycerol polyricinoleate, polyglycerol poly-12-hydroxystearate or polyglycerol dimerate. Likewise suitable are mixtures of compounds from more than one of these classes of substances;
7) adducts of from 2 to 15 mol of ethylene oxide with castor oil and/or hardened castor oil;
8) partial esters based on linear, branched, unsaturated or saturated C6/22 fatty acids, ricinoleic acid and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose);
9) mono-, di- and trialkyl phosphates, and mono-, di- and/or tri-PEG-alkyl phosphates and the salts thereof;
10) wool wax alcohols;
11) polysiloxane/polyalkyl polyether copolymers and corresponding derivatives;
12) mixed esters of pentaerythritol, fatty acids, citric acid and fatty-alcohol as disclosed in DE 1165574 and/or mixed esters of fatty acids having 6 to 22 carbon atoms, methylglycose and polyols, preferably glycerol or polyglycerol and
13) polyalkylene glycols.

The adducts of ethylene oxide and/or of propylene oxide with fatty alcohols, fatty acids, alkylphenols, glycerol monoesters and diesters, and sorbitan monoesters and diesters of fatty acids or with castor oil are known, commercially obtainable products. They are mixtures of homologs whose average degree of alkoxylation corresponds to the ratio of the amounts of ethylene oxide and/or propylene oxide and substrate with which the addition reaction is carried out. C₁₂/C₁₈ fatty acid monoesters and diesters of adducts of ethylene oxide with glycerol are disclosed in DE 2024051 as refatting agents for cosmetic preparations. C₈/C₁₈ Alkyl monoglycosides and oligoglycosides, their preparation and their use are known in the prior art. They are prepared in particular by reacting glucose or oligosaccharides with primary alcohols having 8 to 18 C atoms. With regard to the glycoside ester, both monoglycosides in which a cyclic sugar residue is glycosidically linked to the fatty alcohol, and oligomeric glycosides having a degree of oligomerization of up to, preferably about 8 are suitable. The degree of oligomerization is a statistical average which is based on a homolog distribution usual for such technical products.

Zwitterionic surfactants can also be used as emulsifiers. Zwitterionic surfactants refer to those surface-active compounds which have in the molecule at least one quaternary ammonium group and at least one carboxylate and one sulfonate group. Particularly suitable zwitterionic surfactants are the so-called betaines such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxylmethyl-3-hydroxyethylimidazolines having in each case 8 to 18 C atoms in the alkyl or acyl group, and cocoacylaminoethylhydroxyethylcarboxymethyl glycinate.

The fatty amide derivatives known under the CTFA name Cocamidopropyl Betaine is particularly-preferred. Ampholytic surfactants are likewise suitable emulsifiers. Ampholytic surfactants mean those surface-active compounds which, apart from a C₈/C₁₈ alkyl or acyl group, comprise in the molecule at least one free amino group and at least one —COOH— or —SO₃H group and are able to form inner salts. Examples of suitable ampholytic surfactants are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 C atoms in the alkyl group. Particularly preferred ampholytic surfactants are N-cocoalkylaminopropionate, cocoacylaminoethylaminopropionate and C₁₂/C₁₈ acylsarcosine. Besides ampholytic emulsifiers, quaternary ones are also suitable, with particular preference for those of the ester quat type, preferably methyl-quaternized difatty acid triethanolamine ester salts.

The amount of surface-active agents in the suspensions of the invention is in the range from 0.1 to 10% by weight, preferably 0.5 to 8% by weight, particularly preferably from 1 to 5% by weight, based on the total amount of the suspensions.

Examples of suitable desiccants are compounds selected from the group consisting of alkali metal and alkaline earth metal sulfates such as sodium, calcium and magnesium sulfates, alkali metal and alkaline earth metal chlorides such as sodium, calcium and magnesium chlorides, and silica gel. CaCl₂ is to be mentioned as very particularly preferred desiccant.

The amount of desiccant employed is generally between 0.1 and 20% by weight, preferably between 0.5 und 15% by weight, particularly 15 preferably between 1.0 and 10% by weight, based on the total amount of the suspension.

The invention also relates to a process for preparing the suspensions described at the outset, which comprises
a) grinding solid particles of one or more salts of ascorbic acid in a dispersant in which the salts of ascorbic acid are insoluble until the average particle size is from 0.01 to 1000 μm, it being possible to add the oxidation-sensitive substance(s) selected from the group consisting of carotenoids, retinoids and unsaturated fatty acids to the dispersant before, during or after the grinding, or
b) grinding solid particles of one or more salts of ascorbic acid without using a continuous phase until the average particle size is from 0.01 to 1000 μm, and then suspending the ground particles in a dispersant in which the salts of ascorbic acid are insoluble, it being possible to add the oxidation-sensitive substance(s) selected from the group consisting of carotenoids, retinoids and unsaturated fatty acids to the dispersant before, during or after the suspending of the solid ascorbate particles.

The grinding in a) can take place in a manner known per se, e.g. 40 using a bead mill. Depending on the type of mill used, this entails grinding until the particles have the abovementioned average particle size D[4.3], determined by Fraunhofer diffraction, of from 0.01 to 1000 μm, preferably from 0.1 to 500 μm, particularly preferably from 0.2 to 200 μm, very 45 particularly preferably in the range from 0.5 to 50 μm, especially 0.5 to 20 μm.

Further details of grinding and the apparatus employed therefor are to be found inter alia in Ullmann's Encyclopedia of Industrial Chemistry, Sixth Edition, 1999, Electronic Release, Size Reduction, chapter 3.6.: Wet Grinding.

A preferred embodiment of the wet grinding described in a) takes place in the presence of one or more thickeners, the oxidation-sensitive substance(s) being added to the dispersant after grinding.

Besides wet grinding it is also possible to prepare the suspensions of the invention as in b) by dry grinding the solid particles of one or more salts of ascorbic acid and subsequently suspending the ground particles in a dispersant in which the salts of ascorbic acid are insoluble. Dry grinding means in this connection grinding without the use of a continuous phase. The oxidation-sensitive substance(s) selected from the group consisting of carotenoids, retinoids and unsaturated fatty acids can be added to the dispersant before, during or after the suspending of the solid ascorbate particles.

A preferred embodiment of the preparation of the suspensions described under b) takes place in such a way that the oxidation-sensitive substance(s) are dissolved or dispersed in a dispersant even before addition of the ascorbate particles.

Further details of dry grinding are to be found inter alia in Ullmann's Encyclopedia of Industrial Chemistry, Sixth Edition, 1999, Electronic Release, Size Reduction, chapter 3.4.

The invention also relates to the use of solid particles of one or more salts of ascorbic acid as antioxidants for oxidation-sensitive substances selected from the group consisting of carotenoids, retinoids and unsaturated fatty acids in a dispersant in which the salts of ascorbic acid are insoluble.

The use according to the invention advantageously takes place using solid particles of one or more salts of ascorbic acid having an average particle size in the range from 0.01 to 1000 μm, preferably from 0.1 to 500 μm, particularly preferably from 0.2 to 200 μm, very particularly preferably in the range from 0.5 to 50 μm, especially 0.5 to 20 μm.

The use which includes α-tocopherol as additional, oil-soluble antioxidant is furthermore preferred.

The suspensions of the invention are distinguished inter alia in that it is possible to dispense with the use of protective gas during the production, bottling and storage thereof while simultaneously ensuring adequate stability.

Adequate stability means for the purposes of the invention that the recovery of the retinoid in the preparation after storage at 40° C. for at least 2 weeks is at least 90%. In addition, no unwanted color changes occur during storage of the preparations of the invention.

The following table shows that the suspensions of the invention using solid particles of salts of ascorbic acid, particularly using solid sodium ascorbate particles, have advantageous properties. Whereas retinol preparations using solid ascorbic acid particles as disclosed in EP-B-590 029 show a significant reduction in retinol after storage for only two weeks, the retinol recovery rate in the presence of solid sodium ascorbate particles—with and without protective gas—is more than 90%.

| | Content in % by weight | | | | Recovery rate in % | | |
|---|---|---|---|---|---|---|---|
| | | Sodium | Ascorbic | Toco- | after x weeks | | |
| Test | Retinol | ascorbate | acid | pherol | x = 0 | x = 1 | x = 2 |
| 1^{a)} | 3.0 | — | 24.0 | — | 100 | 91.1 | 89.1 |
| 2^{a)} | 3.0 | 24.0 | — | — | 100 | 96.3 | 96.7 |
| 3^{a)} | 3.0 | — | 24.0 | 6.0 | 100 | 94.6 | 90.6 |
| 4^{a)} | 3.0 | 24.0 | — | 6.0 | 100 | 97.0 | 96.3 |
| 5^{b)} | 3.0 | — | 24.0 | — | 100 | 75.6 | 64.5 |
| 6^{b)} | 3.0 | 24.0 | — | — | 100 | 96.4 | 96.4 |
| 7^{b)} | 3.0 | — | 24.0 | 6.0 | 100 | 81.5 | 72.5 |
| 8^{b)} | 3.0 | 24.0 | — | 6.0 | 100 | 98.0 | 94.1 |
| 9^{c)} | 3.0 | — | 24.0 | — | 100 | 94.6 | 87.7 |
| 10^{c)} | 3.0 | 24.0 | — | — | 100 | 97.3 | 92.9 |
| 11^{c)} | 3.0 | — | 24.0 | 6.0 | 100 | 95.1 | 87.9 |
| 12^{c)} | 3.0 | 24.0 | — | 6.0 | 100 | 97.9 | 95.5 |
| 13^{d)} | 3.0 | — | 24.0 | — | 100 | 75.0 | 61.3 |
| 14^{d)} | 3.0 | 24.0 | — | — | 100 | 97.2 | 91.5 |
| 15^{d)} | 3.0 | — | 24.0 | 6.0 | 100 | 75.5 | 52.0 |
| 16^{d)} | 3.0 | 24.0 | — | 6.0 | 100 | 96.0 | 91.4 |

Storage temperature: 23° C.^{a)}, 40° C.^{b)}, under protective gas in aluminum bottles
Storage temperature: 23° C.^{c)}, 40° C.^{d)}, without protective gas in aluminum bottles

The suspensions are suitable inter alia as additive to human food and animal feed preparations and compound feeds, as compositions for producing pharmaceutical and cosmetic preparations, and for producing dietary supplement products in the human and animal sectors.

The suspensions can preferably be employed for the preparation of cosmetic preparations.

The suspensions can also preferably be employed as feed additive in animal nutrition, especially for applying or spraying onto animal feed pellets.

The use as animal feed additive takes place in particular by directly spraying on the suspensions of the invention, where appropriate after dilution with oils, for example on animal feed pellets as so-called post-pelleting application.

A preferred embodiment of the spraying process consists of loading the animal feed pellets with the oily suspension under reduced pressure.

Examples thereof are to be found inter alia in GB-A-2 232 573 and in EP-A-0 556 883.

Typical areas of use in the human food sector are, for example, the vitaminization of beverages, dairy products such as yoghurt, milk drinks or milk ice, and blancmange powders, egg products, baking mixes and confectionery.

In the cosmetics sector, the oily suspensions can be used for example for vitamin-containing body care compositions, for example in the form of a cream, of a lotion, as lipsticks or makeup.

The invention further relates to dietary supplements, animal feeds, human foods and pharmaceutical and cosmetic preparations comprising the oily suspensions of water-soluble vitamins described at the outset.

Dietary supplement products and pharmaceutical preparations comprising the suspension of the invention mean, inter alia, tablets, coated tablets and, preferably, hard and soft gelatin capsules.

Cosmetic preparations which may comprise the suspensions of the invention are, for example, preparations whch can be applied topically, in particular decorative body care compositions such as lipsticks, facial makeup in the form of a cream, and lotions.

The preparation of the suspensions of the invention is explained in detail in the following examples.

### EXAMPLE 1

A mixture of 200 g of sodium L-ascorbate, 100 g of all-rac-α-tocopherol and 98 g of all-trans-retinol in 550 g of a medium chain-length triglyceride (Delios® SK from Grünau, Germany) was stirred with a paddle stirrer until a homogeneous suspension was obtained. The mixture was then transferred into a stirrable receiver from which the suspension was conveyed by means of a tubing pump through a continuously operated bead mill (Dyno Mill KDL Spezial). The grinding receptacle of the bead mill was charged with 400 g of glass beads (diameter 800 to 1200 μm). The fine-particle suspension emerging from the mill was collected and measured using a particle sizer (Malvern Mastersizer). The grinding process was repeated until 90% of the suspended particles had a particle size of less than 10 μm [D(0.9)<10 μm]. This corresponded to an average particle size D[4.3] of 5.2 μM.

### EXAMPLE 2

A mixture of 400 g of sodium L-ascorbate, 100 g of all-rac-α-tocopherol, 50 g of all-trans-retinol and 15 g of Tixogel® VP (bentonite from Süd Chemie) in 300 g of a medium chain-length triglyceride (Delios® SK from Grünau, Germany) was stirred with a paddle stirrer until a homogeneous suspension was obtained. The mixture was then transferred into a stirrable receiver from which the suspension was conveyed by means of a tubing pump through a continuously operated bead mill (Dyno Mill KDL Spezial). The grinding receptacle of the bead mill was charged with 400 g of glass beads (diameter 800 to 1200 μm). The fine-particle suspension emerging from the mill was collected and measured using a particle sizer (Malvern Mastersizer). The grinding process was repeated until 90% of the suspended particles had a particle size of less than 20 μm [D(0.9)<20 μm]. This corresponded to an average particle size D[4.3] of 10.9.

After removal from the grinding elements, a portion of the dispersion was diluted with 10 times the amount of the oil used and left to stand for 12 h. Neither the undiluted nor the diluted dispersion showed signs of sedimentation over this period.

### EXAMPLE 3

A mixture of 100 g of sodium-L-ascorbate, 100 g of all-rac-α-tocopherol, 50 g of all-trans-retinol and 15 g of Tixogel® VP (bentonite from Süd Chemie) in 300 g of a medium chain-length triglyceride (Delios® SK from Grünau, Germany) was stirred with a paddle stirrer until a homogeneous suspension was obtained. The mixture was then transferred into a stirrable receiver from which the suspension was conveyed by means of a tubing pump through a continuously operated bead mill (Dyno Mill KDL Spezial). The grinding receptacle of the bead mill was charged with 400 g of glass beads (diameter 800 to 1200 μm). The fine-particle suspension emerging from the mill was collected and measured using a particle sizer (Malvern Mastersizer). The grinding process was repeated until 90% of the suspended particles had a particle size of less than 20 μm [D(0.9)<20 μm]. This corresponded to an average particle size D[4.3] of 10.9 μm.

### EXAMPLE 4

A mixture of 200 g of sodium L-ascorbate, 200 g of all-rac-α-tocopherol and 10 g of Cremophor® CO 40 (from BASF) in 255 g of a medium chain-length triglyceride (Miglyol® from Hüls, Germany) was stirred with a paddle stirrer until a homogeneous suspension was obtained. The mixture was then transferred into a stirrable receiver from which the suspension was conveyed by means of a tubing pump through a continuously operated bead mill (Dyno Mill KDL Spezial). The grinding receptacle of the bead mill had been charged with 400 g of glass beads (diameter 800 to 1200 μm). The fine-particle suspension emerging from the mill was collected and measured using a particle sizer (Malvern Mastersizer). The grinding process was repeated until 90% of the suspended particles had a particle size of less than 20 μm [D(0.9)<20 μm]. This corresponded to an average particle size D[4.3] of 10.9 μm. The suspension was then admixed with a solution of 50 g of all-trans-retinol in 285 g of Miglyol and again stirred with a paddle stirrer until a homogeneous suspension was obtained.

### EXAMPLE 5

200 g of sodium ascorbate comminuted to a particle size of <10 μm by dry grinding were added to a mixture of 200 g of all-rac-α-tocopherol, 50 g of all-trans-retinol and 10 g of Cremophor® CO 40 (from BASF) in 540 g of a medium chain-length triglyceride (Miglyol® from Hüls, Germany.). This mixture was then stirred with a paddle stirrer until a homogeneous suspension was obtained.

1. A suspension comprising
A) at least one oxidation-sensitive substance selected from the group consisting of carotenoids, retinoids and unsaturated fatty acids and
B) solid particles of one or more salts of ascorbic acid in a dispersant wherein said one or more salts of ascorbic acid are insoluble.

2. A suspension as claimed in claim 1, comprising
A) 0.1 to 40% by weight of one or more oxidation-sensitive substances selected from the group consisting of carotenoids, retinoids and unsaturated fatty acids and
B) 1 to 50% by weight of one or more salts of ascorbic acid,
where the % by weight are based on the total amount of said suspension.

3. A suspension as claimed in claim 1, comprising
A) at least one retinoid and
B) solid particles of one or more alkali metal and/or alkaline earth metal salts of ascorbic acid.

4. A suspension as claimed in claim 1, comprising said solid particles of one or more salts of ascorbic acid wherein the average particle size of said solid particles of one or more salts of ascorbic acid is in the range from 0.01 to 1000 μm.

5. A suspension as claimed in claim 1, comprising retinol and at least one alkali metal and/or alkaline earth metal salt of ascorbic acid.

6. A suspension as claimed in claim 5, comprising sodium ascorbate and retinol.

7. A suspension as claimed in claim 1, further comprising α-tocopherol.

8. A suspension as claimed in claim 1, wherein said dispersant is an oil capable of being utilized in cosmetics and in human or animal nutrition.

9. A suspension as claimed in claim 1, further comprising at least one desiccant, at least one thickener, and/or at least one surface-active agent, or mixtures thereof.

10. A process for preparing said suspension as claimed in claim 1, which comprises
a) grinding solid particles of one or more salts of ascorbic acid in a dispersant until the average particle size is from 0.01 to 1000 μm, wherein said one or more salts of ascorbic are insoluble in said dispersant, and
optionally adding said one or more oxidation-sensitive substance(s) selected from the group consisting of carotenoids, retinoids and unsaturated fatty acids to the dispersant before, during or after said grinding, or
b) grinding solid particles of one or more salts of ascorbic acid until the average particle size is from 0.01 to 1000 μm, then
suspending the ground particles in a dispersant wherein said one or more salts of ascorbic acid are insoluble, and optionally adding said one or more oxidation-sensitive substance(s) selected from the group consisting of carotenoids, retinoids and unsaturated fatty acids to the dispersant before, during or after the suspending of the solid ascorbate particles.

11. A method of reducing oxidation of one or more oxidation-sensitive substances selected from the group consisting of carotenoids, retinoids and unsaturated fatty acids, said method comprising mixing solid particles of one or more salts of ascorbic acid with said one or more oxidation-sensitive substances.

12. The method as claimed in claim 11, wherein the average particle size of said solid particles is in the range from 0.01 to 1000 μm.

13. The method as claimed in claim 11, further comprising mixing α-tocopherol.

14. A method of reducing oxidation of a human food, an animal feed, a pharmaceutical preparation or a cosmetic preparation, said method comprising mixing said suspension as claimed in claim 1 with said human food, said animal feed, said pharmaceutical or said cosmetic preparation.

15. The method as claimed in claim 14, wherein said suspension is mixed with said animal feed.

16. The method as claimed in claim 15, wherein said animal feed is in the form of one or more pellets.

17. The method as claimed in claim 16, said one or more pellets are mixed with said suspension under a reduced pressure.
